(19) United States
(12) Patent Application Publication
(10) Pub. No.: US 2023/0149111 A1
(43) Pub. Date: May 18, 2023

(54) Device for Foreskin Restoration

(71) Applicant: Tamir Levy Lomholt, Zichron Ya'acov (IL)

(72) Inventor: Tamir Levy Lomholt, Zichron Ya'acov (IL)

(21) Appl. No.: 17/981,503

(22) Filed: Nov. 7, 2022

Related U.S. Application Data
(60) Provisional application No. 63/279,193, filed on Nov. 15, 2021.

(51) Int. Cl. A61B 90/00

(52) U.S. Cl. CPC A61B 90/02; A61B 90/08

(57) ABSTRACT

The present disclosure provides a foreskin restoration device, and related methods of use, which apply an adjustable bidirectional tension to the foreskin of a user as well as provide the capability to selectively orient the skin-contacting elements with respect to the direction of the shaft to compensate for irregularities in skin length and tautness. This gives the user the maximum versatility in deciding which part of the foreskin to stretch and encourage growth on. In addition, it allows the user to tug at a very high level of tension without hurting his glans. The proposed design is compact, easy to use, comfortable to wear throughout the day, and provides the capability for quick and easy adjustments.

### CROSS REFERENCE TO RELATED APPLICATIONS

This Patent application claims the benefit of provisional application US 63/279,193, filed Nov. 15, 2021.

### FIELD OF INVENTION

The present invention relates generally to foreskin restoration devices. More specifically, the present invention relates to a foreskin restoration device configured to apply adjustable levels of bidirectional tension to the foreskin of a user while providing axial freedom of movement of the skin-contacting elements to account for the different body shapes of users.

### BACKGROUND

Although the idea of restoring foreskin may seem bizarre to some, there are many good reasons for doing so. The foreskin of intact males produces pheromones which have been proven to increase the man’s attractiveness to others.

Removing the foreskin also removes its natural gliding, “lubricating” function. Studies have found that, of women who expressed a preference, 90% favored sex with intact, rather than circumcised, men, and that women are 40% more likely to have multiple orgasms with intact men. The male victim of circumcision also has greatly reduced sexual pleasure as the result of losing his foreskin. Besides losing millions of sensory nerve endings in the cut off skin, his unprotected glans is converted from a moist, glassy smooth, highly sensitive mucoid surface into a rough, dry, cornified structure with greatly reduced sensitivity.

These are some of the many reasons that some men have decided to restore their foreskins, however methods of foreskin restoration presently available are limited.

One method has been the creation of a new foreskin through surgical techniques. Donor skin is usually taken from the scrotum and attached to the penis so as to form a somewhat natural-appearing foreskin. The procedure is expensive, costing several thousand dollars. It has all the risks associated with surgery, including scarring, infection, hemorrhage, and risk of reactions to anesthesia. The transplanted skin also does not have the characteristics of normal foreskin: It is wrinkled rather than smooth, has hair growing where it doesn’t belong, and, being deprived of nerves, it lacks sensation. After surgery, there is a period of recovery during which the man cannot engage in sex. But the greatest disadvantage is that the surgically restored foreskin does not have the normal gliding action of the natural foreskin.

Organizations interested in foreskin restoration are unanimous in their recommendation that restoration be achieved through the application of tension to the skin of the penile shaft. Tension on skin does not merely stretch the skin, but, more importantly, it actually induces growth of new skin tissue.

Foreskin restoration through tension avoids the disadvantages of surgery and can produce a foreskin which is nearly normal in both appearance and function. But, as generally employed, restoration through tension is very time consuming, generally taking several years to complete. It also has many other disadvantages, to be described below.

At present, in the market there are restoration devices that use 4 main methods of applying tension to the skin. All of these devices connect to the skin with the assembly of a dome or a conical shaped base, where the skin is being pulled up to, and a gripping silicon cone on top to hold the skin in place.

### Method 1: Devices That Uses Air Pressure

These devices blow air into the foreskin tunnel until the foreskin expands like a balloon and thus creates tension to the skin. The disadvantages of this method are that air tends to release thus the user needs to add pressure frequently. Also, in order to prevent air from entering into the urethra a small piece of tape needs to be applied to cover the opening. Needless to say that this tape should be removed each time one needs to urinate and a new piece of tape should be applied before using the device again. Avoiding using the tape could be risky due to air going into the urethra and could cause pain and discomfort for the user.

### Method 2: Devices That Stretches the Skin With Weight

These devices tend to be large and uncomfortable to walk with due to the heavy weight that swings along with every step. Due to their size, they are visible through clothing, therefore might not be suitable for use in public if one wants discretion.

### Method 3: Devices That Stretch the Skin With an Elastic Strap

One end of the strap is connected to the tip of the device and the other end is connected whether to the knee, the foot, around the shoulder, or any other part of the body, either by clamping to a sock or a knee bandage or by creating a loop around the body part the user wishes to attach the strap to. The main disadvantage of this method is the fact that a long strap is attached to the body and could limit one’s movements. The strap could also be visible if wearing shorts and thus a problem if one is seeking to stretch his foreskin in a discreet manner. Another disadvantage is the difficulty of controlling the amount of tension applied to the skin if one is moving or changing body position.

### Method 4: Dual Tension \ Bi-directional Tension Devices

These devices use a pusher head that pushes the glans towards the body on one end, and on the other end the skin is being pulled away from the body and thus creating tension. The tension can be applied and maintained by elastic bands, a locking screw or a spring.

When using elastic bands, it is difficult to adjust and aim the amount of tension. The device needs to be long in order to create a sufficient distance for the elastic band to be stretched and thus create tension on the skin.

When using a lock screw, the tension is fixed and non-dynamic, so if for example one is applying tension while standing, when he’ll be sitting the device will no longer stretch the skin sufficiently due to the change in body position.

When using a spring the amount of tension is determined by the spring’s force and cannot be changed unless the spring is changed altogether. The distance between the two ends of the Bi-directional device that is enough to create tension varies so much throughout the day due to different factors such as the amount of skin being pulled onto the device.

Another disadvantage that is common to all of the tension devices is the fact that the skin-contacting elements can go only up and down along the axial direction of the central rod of the device, with no freedom of movement to account for the shape and angle of the glans or differences in skin length and tautness. This means that when the user starts to pull the skin along the rod, he will stop once the shortest part of the skin is stretched to the maximum. If there are other parts of the skin which are longer, they will not be stretched sufficiently. Many men have differences in their skin’s length and tautness on different parts of their remaining foreskin. Also, the skin’s stretchable length is determined by the way the user applied the skin to the stretching device. Since a minor difference in length could create a big difference in tension level, achieving an equal amount of tension to all parts of the foreskin could be very difficult to achieve with the existing devices today on the market.

It is within this context that the present invention is provided.

### SUMMARY

The present disclosure provides a foreskin restoration device, and related methods of use, which apply an adjustable bidirectional tension to the foreskin of a user as well as provide the capability to selectively orient the skin-contacting elements with respect to the direction of the shaft to compensate for irregularities in skin length and tautness. This gives the user the maximum versatility in deciding which part of the foreskin to stretch and encourage growth on. In addition, it allows the user to tug at a very high level of tension without hurting his glans. The proposed design is compact, easy to use, comfortable to wear throughout the day, and provides the capability for quick and easy adjustments.

According to a first aspect of the present disclosure, there is provided a foreskin restoration device, comprising: a central rod having an axis oriented along a first direction; a pushing element coupled to a first end of the rod, the pushing element having a concave area shaped to contact the glans; a locking piece affixed about the rod and which is moveable along the length of the rod.

The device further comprises a body, the body being configured to slide freely along the length of the rod between the pushing element and the locking element, the body being further configured to connect to the locking element, wherein the body and locking element become fixed in place at a point along the length of the rod when connected; and a skin gripping assembly, the skin gripping assembly comprising an inner bell-shaped silicone element and an outer bell-shaped silicone element arranged concentrically with one another such that the foreskin of a user may be held between the two and each having an opening through which the rod is threaded, with the opening diameter being greater than the rod diameter.

The skin gripping assembly is positioned between the pushing element and the body, and is coupled to the body by a plurality of adjustable elastic connections distributed radially about the rod, such that adjustment of the elastic connections allows the tension applied by the gripping assembly to the foreskin and the orientation of the skin gripping assembly to change with respect to the first direction according to a user’s preference.

In some embodiments, the elastic connections are elastic loops which are threaded through the body.

In some embodiments, the device further comprises a securing element through which the rod is threaded, and which is positioned on the opposing side of the ball-shaped silicone elements, the elastic connections having splayed ends which are hooked into corresponding grooves of the securing element.

In some embodiments, the body has a plurality of grooves through which the elastic connections are threaded.

The exterior of the body may in such cases also have a plurality of hooking points which the opposing ends of the elastic connections can be secured to in order to adjust the tension and orientation applied by the skin gripping assembly.

In some embodiments, the rod is threaded, and the locking element comprises a threaded inner surface that engages with the rod thread so that the position of the locking element along the length of the rod is controlled by clockwise and anti-clockwise rotation.

The locking element may then further comprises a double-sided threaded heat insert for engaging with a threaded insert at the proximal end of the body to secure the two parts together.

In some embodiments, the body comprises an opening through which the rod is threaded, the opening having a greater diameter than the rod.

In some embodiments, the rod is made of stainless steel.

### BRIEF DESCRIPTION OF THE DRAWINGS

Various embodiments of the invention are disclosed in the following detailed description and accompanying drawings.

FIG. 1 illustrates an isometric view of an example configuration of the disclosed device. The elastic connections between the body and the gripping assembly are in a position to apply maximum tension.

FIG. 2 illustrates an isometric view of the example configuration of the disclosed device with the bell-shaped elements of the gripping assembly made transparent to show the securing element for the elastic connections. The elastic connections between the body and the gripping assembly are in a position to apply minimum tension.

FIG. 3 illustrates an isometric view of the example configuration of the disclosed device. The elastic connections between the body and the gripping assembly are being manipulated to reorient the gripping assembly with respect to the axial direction of the rod.

FIG. 4 illustrates an isometric view of the example configuration of the disclosed device with the bell-shaped elements of the gripping assembly having been pulled back into a reverse position to show the securing element for the elastic connections.

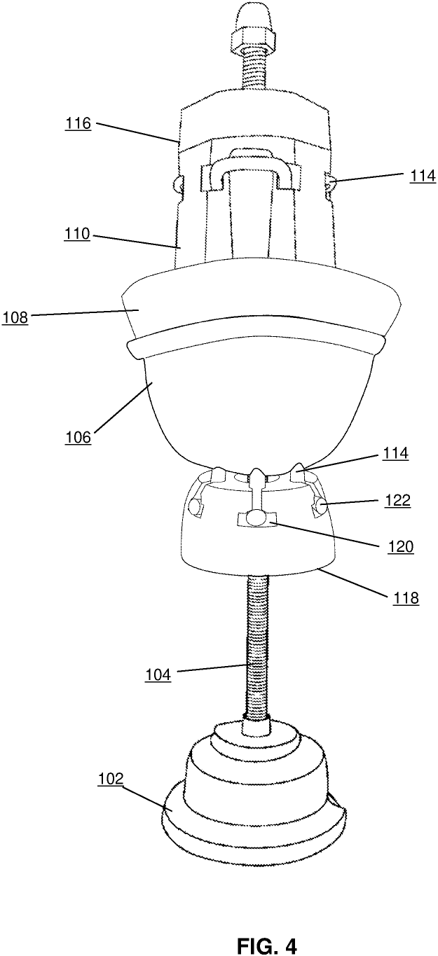

FIG. 5 illustrates an isometric view of the example configuration with the outer silicone gripping element pulled back in a position ready for the user to place their foreskin on the inner bell.

FIG. 6 illustrates an isometric view of an example configuration of a coupling between the locking element and the body of the device of the present disclosure.

FIG. 7 illustrates an isometric view of the example configuration of the device of the present disclosure viewed from the opposing end to the pushing element.

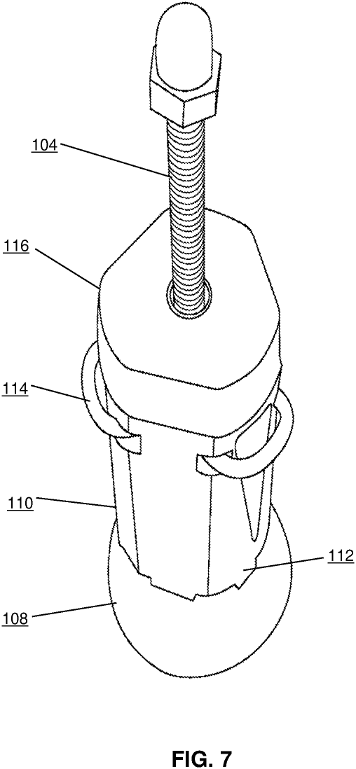

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. Numerical quantities given in this description are approximate unless stated otherwise, meaning that the term “about” or “approximately” can be inferred when not expressly stated.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments.

The terminology used herein is for the purpose of describing particular embodiments only and is not intended to be limiting. As used herein, the singular forms “a,” “an” and “the” are intended to include the plural forms as well, unless the context clearly indicates otherwise.

Spatially relative terms, such as “under,” “below,” “lower,” “over,” “upper” and the like, may be used herein for ease of description to describe one element or feature’s relationship to another when the apparatus is right side up.

The terms “first,” “second,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

A technical problem solved by the present disclosure is to provide a foreskin restoration device with a configuration that enables an “Adjustable tugging system” (A.T.S), facilitated by a skin gripping assembly. The proposed device will give the user an equal all-around stretch to the foreskin. With its unique A.T.S combined with the double silicone layers for holding the foreskin between, the user will enjoy a long and consistent stretch without slippage of the skin. Its soft silicone pushing element will allow the user to tug comfortably for hours every day.

The proposed assembly as per its preferred embodiments is simple to handle. Its design allows the user to enjoy a flexible long stretch with a short rod and its smooth surface and small size makes it unfelt between the legs even with tight jeans. Furthermore, each part that comes in touch with the penile skin may be made of medical grade soft silicone for maximum comfort.

The configuration of the device allows the user to determine exactly which part of the foreskin will be stretched. By applying more tension to a specific part of the foreskin, thus encouraging more skin growth to that specific area, the user can fix an asymmetrical foreskin and achieve a better looking final result.

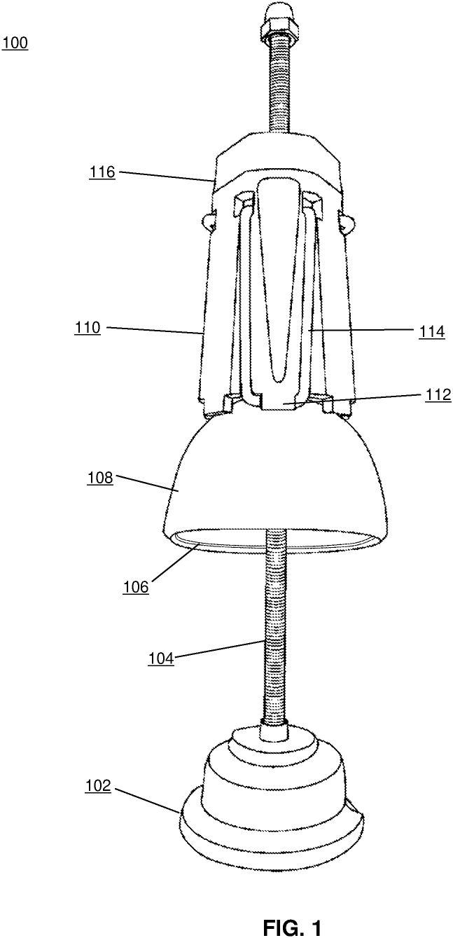

Referring to FIG. 1, an isometric view of an example configuration of the disclosed device 100 is shown.

As can be seen, the device comprises a rod, in this case a threaded stainless steel rod 104, which runs centrally through all its components. At the base of the rod 104 is a soft silicone pushing element 102 that is configured to push the glans towards the scrotum once the foreskin of a user is held between the silicone bell-shaped elements 106 and 108. Together, these tugging and gripping elements make a firm yet comfortable grip.

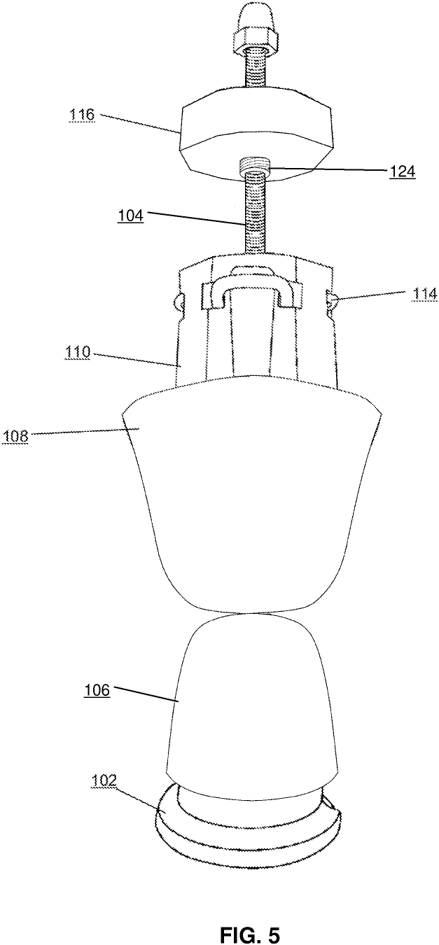

The first step in applying the device is to aim the pusher 102 to face and touch the glans. The device should have the flexible outer bell-shaped element 108 pulled back, revealing the inner bell-shaped element 106 (see FIG. 5). Then with the other hand the skin of the penis shaft is pulled forward onto the base of the inner element 106. Next step is to return the silicone gripper 108 into its shown position so that it falls on the skin, securing it in place between the two silicones layers. Once the skin of the penis shaft is secured between the two bell-shaped silicone elements, the bioplastic body 110 can be pulled along the rod 104 and coupled to the locking element 116, securing it at a chosen point along the rod 104 to apply a constant tension to the skin.

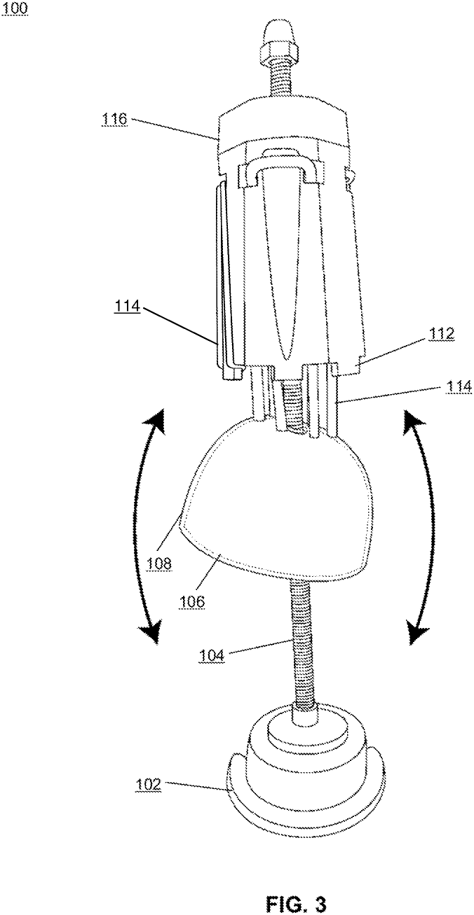

The whole gripping assembly has a hole in the top centre with a larger diameter than the diameter of the rod 104 in order to allow it to move in a diagonal direction with different angles around the rod. See FIG. 3.

The assembly is secured to the bioplastic body 110, for example with three loops of a high-quality elastic cord 114.

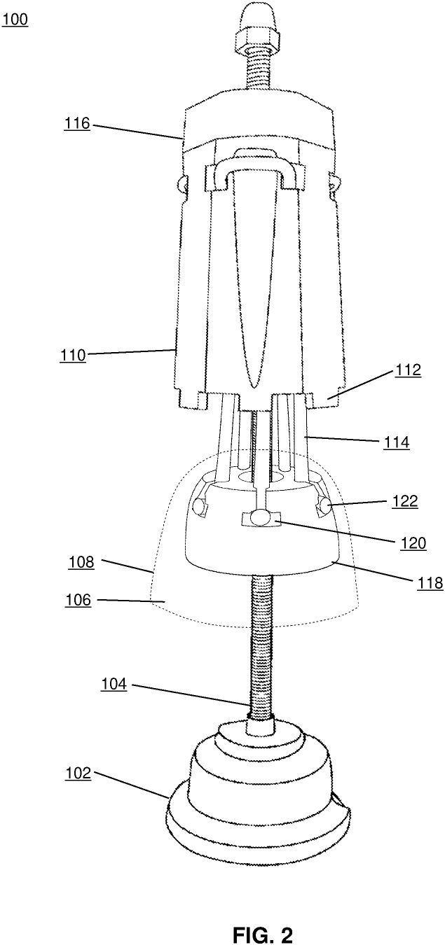

Turning to FIG. 2, an example configuration for anchoring the loops 114 is shown which uses a securing element 118 placed in the concave area of the bell-shaped silicone gripping assembly. This securing element 118 has a plurality of grooves 120 that the ends 122 of the cords can be hooked into, passing through holes in the silicone bells. The opening in the centre of the securing element 118 is also larger than the diameter of the rod so that the direction of the gripping assembly can be oriented with respect to the axial direction of the rod by adjusting the respective tension on the elastic connecting cords 114.

The opposing ends of the cords 114 run through internal channels in the bioplastic body 110 and out through openings at the top. They can then be looped around and hooked on various protrusions 112 at the bottom of the body 110, the tension provided by each cord 114 being dependent on which protrusion it is hooked on.

In FIG. 2, the cords 114 are not hooked on anything, so there is little tension being applied to the gripping assembly on any side. In FIG. 1 all of the cords are hooked to a protrusion, providing maximum tension on every side.

By pulling the ATS assembly upwards along the threaded stainless-steel rod a tension is created. At a user-defined point on the rod, there’s the locker: a cap that can be attached to the ATS body to keep it in place and sustain the tension. The locker has a double-sided threaded heat insert. The interior of the insert is threaded to fit the threaded rod, the exterior of the insert is threaded to fit the threaded insert that is at the top of the ATS body. By turning the cap to the right or left, the user can aim with high precision the total amount of desired tension to the skin. Once the desired amount of tension is achieved, the user turns the locker clockwise while keeping the two parts together (the locker and the ATS body) and the two parts screw together thanks to the threaded assembly mentioned above.

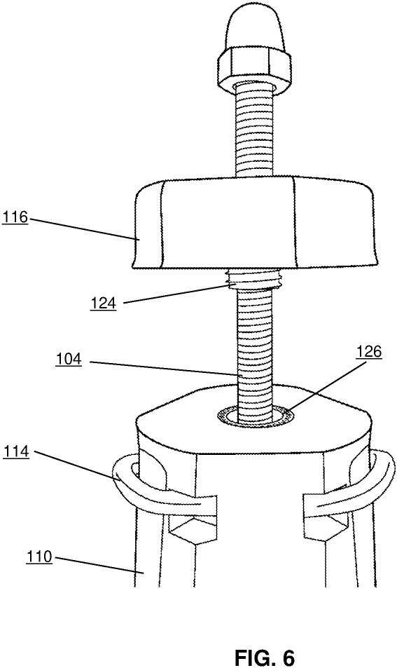

In FIG. 6 the connection between the locking element 116 and the body 110 is shown in more detail.

As can be seen, the locking element couples to the threaded rod 104, and can be moved along the length of the rod by clockwise or anti-clockwise rotation. This is in contrast to the body 110, which has an unthreaded opening through which the rod passes, but a threaded insert 126 at the end proximal to the locking element 116. The locking element has a double sided heat insert 124 which couples to the insert 126 of the body, locking them together.

The effect of this arrangement is that when the locking element 116 is not connected to the body 110, the body 110 can slide freely up and down the length of the rod between the locking element 116 and the gripping assembly, allowing a user to hold the body 110 between their fingers and push the rod 104 downwards, applying pressure to the glans while gripping and thus tugging the held skin in the opposite direction. But when a user wishes to set a predefined tension on their skin, they can adjust the body and the gripping assembly to desired positions then coupled the locking element 116 to the body 110 to stop the free sliding to the body along the shaft and hold it in place at the desire position.

The tugger will adapt its position and angle according to the skin’s length due to being detached from the rod. Combined with the flexibility of the elastic cords, every part of the foreskin will be stretched in a dynamic way, meaning that whether the user is in a constant movement or in a static standing/sitting position, the device will continue to apply a flexible and steady tension to skin.

If the user wants to put more tension on a specific part of his foreskin, he can grab one of the elastic loops, and pull it downwards to hook around a securing protrusion. Then the puller head will turn on its side where he pulled the cords and he will feel how the foreskin gets a stronger stretch in the desired area.

Alternatively, if he wishes to have a very strong stretch to all of the areas of the foreskin, by pulling all of the cords downward and securing them, a very high level of tension is felt without putting more pressure force on the glans. When the user wishes to quickly release the tension, by turning the locker counter-clockwise three times around itself the two parts detach and tension is quickly relieved.

The present disclosure provides an improved form of a foreskin restoration method which allows the user full control to determine the desired amount of tension applied to the skin and to determine exactly the area of the skin the user wishes to apply tension to.

The present disclosure provides a new tugging system based on the dual tension method and a device that will stretch the foreskin with maximum efficiency and comfort.

The present disclosure provides a handheld sophisticated yet simple device to handle. With the claimed device it is very easy to adjust the desired tension level. The pulling head is adjustable according to the skin’s length and position on the device.

The present disclosure provides a device with a smooth surface all around to avoid any discomfort when in contact with the skin. Its small size makes it unfelt between the legs even with tight jeans.

In the disclosed device a relatively short rod is enough for achieving a long, elastic and dynamic stretch. The pulling head can reach up until the tip of the rod and thus maximize it’s potential for stretching distance

The present disclosure provides a device with improved ergonomics. Each part that comes in touch with the penile skin may be made of platinum soft silicone for maximum comfort and quality.

The present disclosure provides a device which may be made from high quality stainless steel, and its 3d printed parts are bioplastic.

The present disclosure provides a smart assembly which provide an ease of use and convenience to the user and is highly portable and can be carried by the user anywhere anytime.

The present disclosure provides a device for achieving a high-tension level on the skin with a reduced pressure on the glans.

The present disclosure provides a universal eco-friendly and an ergonomic design of a handheld foreskin restoration device comprising a restorer and a retainer; a smooth surface all around to avoid any discomfort when in contact with the skin; a soft silicone pusher that pushes the glans towards the scrotum lies at the base of the tension device; above the pusher, there’s the silicone covered dome shaped tugger, when combined with the silicone gripper, together to make a firm yet comfortable grip; a hole on the top center, with a larger diameter than the rod that travels through, to allow it to move in a diagonal direction with different angles around the rod; a tugger attached to the bioplastic body with three loops of a high-quality elastic cord, anchored in six points round the perimeter, to give a wide and equal amount of tension all around; and, a cap termed as locker that can be attached to the ATS cylinder to keep it in place and sustain the tension.

In some examples, by pulling the ATS assembly upwards along the threaded stainless-steel rod, a tension is created. At a user-defined point on the rod, there’s the cap that can be attached to the ATS cylinder to keep it in place and sustain the tension. By turning the cap to the right or left, user can also aim with high precision the total amount of desired tension to the skin.

In some examples, the pulling head adapts its position and angle according to the user’s skin length. Combined with the flexibility of the elastic cords, every part of the foreskin will be stretched in a dynamic way, allowing the tension device to apply a flexible and steady tension to user’s skin regardless of the activity the user is involved in.

In some examples, by applying more tension to a specific part of the foreskin, thus encouraging more skin growth to that specific area, the user can fix an asymmetrical foreskin and achieve a better-looking final result.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the device have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A foreskin restoration device, comprising:
a central rod having an axis oriented along a first direction;
a pushing element coupled to a first end of the rod, the pushing element having a concave area shaped to contact the glans;
a locking piece affixed about the rod and which is moveable along the length of the rod;
a body, the body being configured to slide freely along the length of the rod between the pushing element and the locking element, the body being further configured to connect to the locking element, wherein the body and locking element become fixed in place at a point along the length of the rod when connected; and
a skin gripping assembly, the skin gripping assembly comprising an inner bell-shaped silicone element and an outer bell-shaped silicone element arranged concentrically with one another such that the foreskin of a user may be held between the two and each having an opening through which the rod is threaded, with the opening diameter being greater than the rod diameter;
wherein the skin gripping assembly is positioned between the pushing element and the body, and is coupled to the body by a plurality of adjustable elastic connections distributed radially about the rod, such that adjustment of the elastic connections allows the tension applied by the gripping assembly to the foreskin and the orientation of the skin gripping assembly to change with respect to the first direction according to a user’s preference.

2. A foreskin restoration device according to claim 1, wherein the elastic connections are elastic loops which are threaded through the body.

3. A foreskin restoration device according to claim 1, wherein the device further comprises a securing element through which the rod is threaded, and which is positioned on the opposing side of the ball-shaped silicone elements, the elastic connections having splayed ends which are hooked into corresponding grooves of the securing element.

4. A foreskin restoration device according to claim 1, wherein the body has a plurality of grooves through which the elastic connections are threaded.

5. A foreskin restoration device according to claim 4, wherein the exterior of the body has a plurality of hooking points which the opposing ends of the elastic connections can be secured to in order to adjust the tension and orientation applied by the skin gripping assembly.

6. A foreskin restoration device according to claim 1, wherein the rod is threaded, and the locking element comprises a threaded inner surface that engages with the rod thread so that the position of the locking element along the length of the rod is controlled by clockwise and anti-clockwise rotation.

7. A foreskin restoration device according to claim 6, wherein the locking element further comprises a double-sided threaded heat insert for engaging with a threaded insert at the proximal end of the body to secure the two parts together.

8. A foreskin restoration device according to claim 1, wherein the body comprises an opening through which the rod is threaded, the opening having a greater diameter than the rod.

9. A foreskin restoration device according to claim 1, wherein the rod is made of stainless steel.
